(19) United States
(12) Patent Application Publication
(10) Pub. No.: US 2020/0323929 A1
(43) Pub. Date: Oct. 15, 2020

(54) COMPOSITION OF PROBIOTICS AND DIGESTIVE ENZYMES AND METHOD OF PREPARING AND USING THE SAME

(71) Applicant: Kim SHAFER, Milford, MI (US)

(72) Inventor: Kim SHAFER, Milford, MI (US)

(21) Appl. No.: 16/304,123

(22) PCT Filed: May 24, 2016
(86) PCT No.: PCT/US2016/033976

§ 371 Date: Nov. 21, 2018

(30) Foreign Application Priority Data
May 24, 2016 (US) PCT/US2016/033976

(51) Int. Cl. A61K 35/745; A61K 35/747; A61K 38/46; A61K 38/47; A61K 38/48; A61K 38/54

(52) U.S. Cl. CPC A61K 35/745; C12Y 304/22032; A61K 38/465; A61K 38/47; A61K 38/4873; A61K 38/54; C12Y 301/01003; C12Y 302/01001; C12Y 302/01003; C12Y 302/01004; C12Y 302/01008; C12Y 302/01026; C12Y 302/01108; C12Y 304/22002; A61K 35/747

(57) ABSTRACT

Disclosed are compositions and methods useful for improving blood cholesterol profiles in a mammal, particularly for improving metabolism of cholesterol, for reducing the levels of low-density lipoprotein cholesterol (LDL-C) in the blood, increasing the levels of high-density lipoprotein (HDL-C) in the blood, for improving weight loss in a mammal, and/or for enhancing overall cardiovascular health in a mammal. Methods can involve identifying a mammal in need of lowering of blood LDL-C and/or triglyceride concentrations, and administering to said mammal a specific formulation consisting of a blend of probiotics; specifically, Bifidobacterium infantis, Bifidobacterium bifidum, Lactobacillus acidophilus, Lactobacillus salivarius, Lactobacillus plantarum, Lactobacillus rhamnosus, Bifidobacterium longum, Lactobacillus casei, Lactobacillus paracasei, in combination with a blend of digestive enzymes; specifically, amylase, glucoamylase, lipase, bromelain, maltase, lactase, hemicellulase, xylanase, papain, and invertase. Preferably, the aforementioned probiotics and digestive enzymes are combined into capsules and administered to said mammal three times daily to achieve said lowering of LDL-C and triglyceride concentrations in blood.

### FIELD OF THE INVENTION

The present invention relates to maintaining good health. More specifically, the present invention relates to maintaining good health through the concept of dietary supplements and the use thereof. Further, the inventor has realized advantages of an inventive dietary supplement composition over prior art dietary supplements and has herein disclosed such inventive compositions and methods of making and using, such as improving blood cholesterol profiles in a mammal, particularly for improving metabolism of cholesterol, for reducing the levels of low-density lipoprotein cholesterol (LDL-C) in the blood, and increasing the levels of high-density lipoprotein (HDL-C) in the blood, for improving weight loss in a mammal, and/or for enhancing overall cardiovascular health in a mammal.

Cholesterol is formed primarily in the liver (75% of total) and ingested in the diet, and is generally acknowledged to be very significant as a factor in various disease processes of the human body, including cardiovascular disorders. Cholesterol has a vital role in many physiological processes, including the maintenance of membrane integrity of eukaryotic cells, manufacturing vitamin D in the skin, synthesis of steroid hormones, and formation of neural synapses in the brain Physiological agents that affect cholesterol synthesis and metabolism can be utilized to enhance these processes.

Elevated levels of specific types of cholesterol in the blood can lead to health consequences; including coronary heart disease (CHD), one of the leading causes of death worldwide. Elevated levels of LDL-C and triglycerides (TC) represent risk factors for CHD, whereas high concentrations of plasma high-density lipoprotein cholesterol (HDL) are considered healthy and protective against coronary heart disease (CHD). The cholesterol content per LDL molecule can exhibit a large variation between individuals; therefore, LDL particle size and number can provide independent measures of the risk of CHD. Atherosclerosis is the pathological process that typically underlies CHD morbidity and mortality. This process involves formation of plaques in the intima and media of the arterial wall. These atherosclerotic lesions result from the progressive accumulation of cholesterol and lipids, extracellular matrix material, and inflammatory cells along the arterial walls. The implications of cholesterol in the etiology and prognosis of atherosclerosis and coronary heart disease has stimulated enormous interest in devising novel therapeutic strategies for lowering, or maintaining normal or near-normal, levels of cholesterol in serum. The orally ingested compositions described in this disclosure contain effective amounts of probiotics and digestive enzymes, which reduce or control the concentrations of LDL-C and triglycerides, as well as overall cholesterol levels in the blood, or promote improved cardiovascular function.

Cholesterol is formed primarily in the liver (75% of total) and ingested in the diet, and is generally acknowledged to be very significant as a factor in various disease processes of the human body, including cardiovascular disorders. Cholesterol has a vital role in many physiological processes, including the maintenance of membrane integrity of eukaryotic cells, manufacturing vitamin D in the skin, synthesis of steroid hormones, and formation of neural synapses in the brain. Physiological agents that affect cholesterol synthesis and metabolism can be utilized to enhance these processes.

Elevated levels of specific types of cholesterol in the blood can lead to health consequences; including coronary heart disease (CHD), one of the leading causes of death worldwide. Elevated levels of LDL-C and triglycerides (TC) represent risk factors for CHD, whereas high concentrations of plasma high-density lipoprotein cholesterol (HDL) are considered healthy and protective against coronary heart disease (CHD). The cholesterol content per LDL molecule can exhibit a large variation between individuals; therefore, LDL particle size and number can provide independent measures of the risk of CHD. Atherosclerosis is the pathological process that typically underlies CHD morbidity and mortality. This process involves formation of plaques in the intima and media of the arterial wall. These atherosclerotic lesions result from the progressive accumulation of cholesterol and lipids, extracellular matrix material, and inflammatory cells along the arterial walls. The implications of cholesterol in the etiology and prognosis of atherosclerosis and coronary heart disease has stimulated enormous interest in devising novel therapeutic strategies for lowering, or maintaining normal or near-normal, levels of cholesterol in serum.

### SUMMARY

The disclosure relates to a composition of probiotics and digestive enzymes for improving cholesterol metabolism, for improving overall cardiovascular health, and/or for improving metabolic efficiency or rate to assist with weight control and weight loss in a mammal. The specific formulation of probiotics and digestive enzymes, when used in combination and administered to a mammal, can be utilized to improve the health of a mammal; specifically, for mammals afflicted with hypertension, cardiovascular disease, critical limb ischemia or other disorders related to the vascular system, impaired glucose tolerance, metabolic syndrome, and disorders of the digestive tract. Said formulation of probiotics and digestive enzymes can also be administered to a mammal as a dietary supplement to improve overall wellness of said mammal and of specific organ systems in said mammal in the absence of any specific disease condition as a prophylactic. The present invention is directed toward a dietary supplement composition comprising at least two ingredients within a capsule. The supplement may be developed for human consumption by swallowing of the capsule. The two ingredients may include at least one probiotic ingredient. For example, the composition may include at least two different probiotic ingredients or at least one probiotic ingredient and at least one digestive enzyme. The inventor has realized advantages of this inventive composition over the prior art dietary supplements. The orally ingested compositions described in this disclosure contain effective amounts of probiotics and digestive enzymes, which reduce or control the concentrations of LDL-C and triglycerides, as well as overall cholesterol levels in the blood, or promote improved cardiovascular function.

An object of the invention is to provide a composition that relieves symptoms of irritable bowel syndrome and other digestive diseases, syndromes and illnesses.

An object of the invention is to provide a composition that replaces and replenishes the good bacteria in the human body.

An object of the invention is to provide a composition that supplements the human body's supply of digestive enzymes.

An object of the invention is to provide the inventive composition in a form that has a long shelf the.

An object of the invention is to provide the inventive composition in pill form and to provide a pill that reaches the digestive tract prior to being absorbed.

An object of the invention is to provide a composition that allows users of the composition to ingest foods that otherwise result in adverse reactions by the host body.

An object of the invention is to provide a composition that allows users of the composition to have regular bowel movements.

A preferred embodiment of the present invention involves identifying a mammal in need of lowering of blood LDL-C and/or triglyceride concentrations, and/or raising HDL-C and administering to said mammal a specific formulation consisting of a blend of probiotics; specifically, Bifidobacterium infantis, Bifidobacterium bifidum, Lactobacillus acidophilus, Lactobacillus salivarius, Lactobacillus plantarum, Lactobacillus rhamnosus, Bifidobacterium longum, Lactobacillus casei, Lactobacillus paracasei, in combination with a blend of digestive enzymes; specifically, amylase, glucoamylase, lipase, bromelain, maltase, lactase, hemicellulase, xylanase, papain, and invertase. Identifying a patient in need can be done by any conventional detection method, non-exclusively including blood tests, or identifying and assessing risk factors for cardiovascular disease, such as smoking, drinking, lack of exercise, weight of patient, age, family history, etc. In a preferred embodiment of the aforementioned invention, the aforementioned probiotics and digestive enzymes are combined into capsules and administered to said mammal three times daily to achieve said lowering of LDL-C and triglyceride concentrations in blood.

The above summary of the present invention is not intended to describe each illustrated embodiment, aspect, or every implementation of the present invention.

### BRIEF DESCRIPTION OF THE DRAWINGS

The invention may be more completely understood in consideration of the following description of the invention in connection with the accompanying drawings, in which:

FIG. 1A shows line graphs providing an analysis of a probiotic/digestive enzyme composition in the SHIME® model, with propionate production in the ascending and transverse colon in supplemented vessels (treatment weeks T1, T2, and T3) vs. control vessels (C1 and C2).

FIG. 1B shows a bar graph providing an analysis of a probiotic/digestive enzyme composition in the SHIME® model, with total lactate concentrations (g/L) in supplemented treatment vessels vs controls.

FIG. 1C shows a bar graph providing an analysis of a probiotic/digestive enzyme composition in the SHIME® model, with Quantitative PCR results for the total copies/mL of lactobacilli.

FIG. 1D shows a bar graph providing an analysis of a probiotic/digestive enzyme composition in the SHIME® model, with Quantitative PCR results for the total copies/mL of bifidobacteria.

While the invention is amendable to various modifications and alternative forms, specifics thereof have been shown by way of example in the drawings and will be described in detail. It should be understood, however, that the intention is not necessarily to limit the invention of the particular embodiments described.

### DETAILED DESCRIPTION OF THE INVENTION

The Composition

The invention is directed to a probiotic digestive enzyme dietary supplement and a method of use for humans or other animals, as may be seen in the Figures and as provided herein. The dietary supplement composition (which may also exist as a drug or pharmaceutical) may comprise at least two ingredients. The two ingredients may include at least one probiotic ingredient. For example, the composition may include at least two different probiotic ingredients or at least one probiotic ingredient and at least one digestive enzyme ingredient, or other ingredients in various combinations. In addition, the composition may be substantially, if not completely, devoid of artificial flavors, colorings or preservatives. Further, the supplement may be developed for human or other animal consumption by swallowing or other ingestion technique. In a situation where the composition is developed for consumption by swallowing, the composition may be enclosed within a capsule or other form known to facilitate swallowing.

The terms probiotics, digestive enzymes and dietary supplements have generally accepted definitions. For example, probiotics may be defined as live microorganisms thought to be healthy for the host organism; digestive enzymes may be defined as enzymes that break down polymeric macromolecules into their smaller building blocks in order to facilitate their absorption by the body; dietary supplements may be defined as a preparation intended to supplement the diet and provide nutrients that may be missing or may not be consumed in sufficient quantities in a human's diet.

The probiotic ingredients of the composition may be present in an effective dose. For example, at the time of manufacture, the probiotic ingredients may total at least 6×10⁹ colony forming units (cfu) and may include at least 13×10⁹ cfu of probiotics or more. In a preferred aspect, the probiotic ingredients total at least 13×10⁹ cfu of probiotics. In a more preferred aspect the probiotic ingredients total at least 14×10⁹ cfu of probiotics. A colony forming unit (cfu) is generally accepted as a measure of viable bacterial or fungal numbers. Such quantity of probiotic ingredient may facilitate providing a consumer with an effective dose of probiotics at the time of ingestion, as the inventor has realized that probiotics may be destroyed during storage due to undesirable environments (e.g., temperature extremes) and other reasons.

The probiotic ingredients may comprise a probiotic blend including one or more of the following: Lactobacillus rhamnosus GG, Lactobacillus acidophilus, Lactobacillus casei, Lactobacillus paracasei, Lactobacillus plantarum, Lactobacillus salivarius, Bifidobacterium infantis, Bifidobacterium longum, Bifidobacterium bifidum. In a preferred aspect the composition includes at least one probiotic from each of the strains listed above. Each probiotic ingredient present in the composition may be present in any desired quantity. In one aspect each probiotic ingredient of the composition may be present in an amount at least between 5×1os cfu to 1.5×10⁹ cfu. In a further aspect each probiotic ingredient of the composition may be present in an amount equal to or greater than 1×10⁹ cfu, and in a preferred aspect when combined the nine probiotic ingredients may total as much as, or more than, 13×10⁹ cfu of probiotic ingredients. Preferably the amounts are equal to or are more than 14×10⁹ cfu. In the example, these quantities may be measured at the time of manufacture.

The inventor appreciates that simply introducing a probiotic or probiotics into the GI tract as done in prior instances is not effective due to otherwise poor or undesirable placement of the ingredient within the system and/or lack of an effective dose due to the degradation of the living probiotic ingredient and/or lack of the variety and nature of a desired or sufficient strain or strains of probiotic and/or lack of use of the probiotic blend in combination with the digestive enzyme supplement or supplemental blend (see below regarding enzymes). Further, as the inventor appreciates that lactobacillis acidophilis is a prominent strain of probiotic in the small intestine, and Bifidobacterium bifidum is a prominent strain of probiotic in the large intestine, for instance, it is advantageous to have those supplemental ingredients (and other of the respective supplemental probiotic ingredients noted above and the digestive enzymes noted below) introduced into the GI tract at the appropriate or preferred locations (and in effective amounts). Use of a capsule, such as a vegetable or other capsule that does not immediately release the contents therein (for instance, the capsule delays release beyond the stomach), has a benefit for the positioning of the ingredients within or throughout the GI tract. Use of a blister pack (or other sealing mechanism) for storing the capsule assists in preserving the potency of the ingredients such that the combination of the composition with the capsule in a protected blister package assists with appropriate and effective delivery (location and potency). Further, the particular blend and strains of the respective ingredients, and in the various amounts, have been established by the inventor for desired impact and appropriate delivery.

The digestive enzymes of the composition may be present in an effective dose to supplement existing quantities of enzymes and improve digestion of ingested food and absorption of the nutrients within the ingested food. The digestive enzyme ingredients may comprise any enzyme that is useful in the digestion of ingested food. For example, inventor has developed a particularly effective blend of digestive enzymes comprising some or all of the following: Amylase, Protease, Lipase, Hemicullelase, Invertase, Lactase, Papain, Glucoamylase, Xylanase, and Maltase.

A capsule may enclose the composition to facilitate increasing the shelf-life of the composition, swallowing of the composition, timing a release of the composition after ingestion and other considerations. The capsule may be a gelatin capsule, vegetable-based (e.g., vegetable cellulose) capsule or other type of capsule. If the capsule is a vegetable-based capsule, the capsule may facilitate releasing the probiotics at a desirable location within the digestive tract. Preferably the capsule is a vegetable-based capsule.

An exemplary embodiment of the composition includes a probiotic blend and a digestive enzyme blend enclosed within a vegetable cellulose capsule. The probiotic blend includes 13×10⁹ cfu (116.20 mg) of probiotics consisting of the following species: Lactobacillus rhamnosus GG, Lactobacillus acidophilus, Lactobacillus casei, Lactobacillus paracasei, Lactobacillus plantarum, Lactobacillus salivarius, Bifidobacterium infantis, Bifidobacterium longum, Bifidobacterium bifidum. Preferably each probiotic species is present in quantities of at least 1×10⁹ cfu. In one aspect the digestive enzyme blend includes 272.65 mg of digestive enzymes consisting of the following: amylase, protease, lipase, hemicellulase, invertase, lactase, papain, glucoamylase, xylanase and maltase.

Protecting the composition after manufacture is particularly important as at least the probiotic ingredients may be sensitive to variations in environmental conditions. To facilitate protection of the composition, capsules comprising the composition may be and are preferably stored in blister packs. That is, the blister packs may seal the capsule from a surrounding environment and thus, extend the life of the effective ingredients of the composition.

Any method of using the composition may be used as desired by consumers of the composition. A particularly advantageous program may be to take a single capsule of the composition on a daily basis. Continuous daily use of the composition may result in greater comfort throughout the digestive tract, which may further result in increased energy and general good health of a consumer's body.

Probiotics—Exemplary Ingredients:

Lactobacillus and Species Thereof.

Lactobacillus is a genus within the lactic acid bacteria group, named because the majority of its members convert lactose and other sugars to lactic acid. Lactobacillus may naturally be present in the gastrointestinal tract and other areas of the human body. Lactobacillus is used for treating and preventing diarrhea, including infectious types such as rotaviral diarrhea in children and traveler's diarrhea. It is also used to prevent and treat diarrhea associated with using antibiotics. Other benefits of lactobacillus may include relief from general digestion problems; irritable bowel syndrome (IBS); Crohn's disease; inflammation of the colon; and infection with Helicobacter pylori, the type of bacteria that causes ulcers. Numerous strains of Lactobacillus may be utilized within the composition as exemplary set forth below.

Lactobacillus rhamnosus GG. When administered orally, L. rhamnosus GG adheres to the mucous membrane of the intestine and may help to restore the balance of the GI micro flora; promote gut-barrier functions; diminish the production of carcinogenic compounds by other intestinal bacteria; and, activate the innate immune response and enhance adaptive immunity, especially during infections.

Lactobacillus acidophilus. Within the digestive system, L. acidophilus has been observed as preventing the growth of fungus; thus, helping to prevent infections. Further, L. acidophilus may facilitate lactose digestion in lactose-intolerant subjects and may facilitate the re-colonization of probiotics in the gastrointestinal tract to achieve normal intestinal flora levels.

Lactobacillus casei. L. casei is considered beneficial for the digestive process. It has a wide temperature and pH range meaning it can withstand the acidic environment of the gut. It also promotes L. acidophilus which produces the enzyme amylase. This enzyme assists a person's body in the digestion of carbohydrates, which can help reduce, relieve or prevent conditions such as constipation, lactose intolerance and possibly irritable bowel syndrome.

Lactobacillus paracasei. L. paracasei is a strain of flora (i.e., bacteria) that helps to calm digestive upsets and assists other strains of bacterium. As well, L. paracasei may improve the absorption of nutrients and lipids in the gut.

Lactobacillus salivarius. L. salivarius has been shown to improve bleeding gums, tooth decay, bad breath, thrush and canker sores. In addition, L. salivarius breaks down proteins and produces B vitamins, enzymes and lactic acid.

Lactobacillus plantarum. L. plantarum may create a healthy barrier in a person's colon to keep dangerous bacteria from penetrating the lining of a person's intestines and entering a person's blood stream.

Bifidobacterium and Species Thereof. Bifidobacterium generally reside in the colon and are one of the major genera of bacteria that make up the gut flora. This probiotic may be used to relieve and treat intestinal disorders; may assist in digestion; may be associated with lowering occasions of allergies; and may assist in preventing certain tumor growths. Moreover, the benefits of Bifidobacterium include regulation of intestinal microbial homeostasis, inhibition of pathogens and harmful bacteria that colonize or infect, or both, the gut mucosa, modulate local and systemic immune responses, assist in producing vitamins, and help bioconvert a number of dietary compounds into bioactive molecules. Numerous strains of Bifidobacterium may be utilized within the composition as exemplarily set forth below.

Bifidobacterium longum. B. longum has a high affinity for intestinal colonization. Generally, this probiotic assists in improving the intestinal environment, which may lead to better regularity of bowel movements. Moreover, B. longum assists in maintaining a normal digestive tract, inhibits the growth of harmful bacteria and boosts the immune system.

Bifidobacterium bifidum. B. bifidum helps keep the digestive system running smoothly, blocks the growth of harmful bacteria, and boosts the immune system.

Bifidobacterium Infantis 3 5624 (or other strains). B. infantis may help relieve many of the symptoms associated with irritable bowel syndrome (IBS) in women, including diarrhea and constipation

Digestive Enzyme Facts

Many enzymes and molecules are utilized to break down all the food we eat. In fact, the process of digestion, or the breaking down of large pieces of food, is complex and involves several enzymes, each with a specific function. There is a range of enzymes in our digestive system and each enzyme functions to keep us healthy through breaking down “big” pieces of ingested food into small absorbable molecules.

Stomach Enzymes

The food entering our stomach is made easier to digest by the act of chewing and by the addition of moisture from our saliva and the liquid we drink. The stomach contains several enzymes that work together to partially break down ingested food substances, examples follow:

Amylase. Amylase is an enzyme found in our saliva and in the enzyme blend released from the pancreas. It functions primarily as a starch-dissolving enzyme that takes starch in our food and breaks it down into simple sugars which can be more easily absorbed. Without an enzyme such as Amylase to break down starch, the bacteria residing in our colon would use the starch for food, resulting in bacterial overgrowth, bloating and gas. Preferred amounts of amylase for the teachings herein include 1.33 mg or between 0.5-2.5 mg.

Lipase. This enzyme works throughout the digestive process to break down the fats in our diet. It works with the bile salts excreted from the liver to emulsify and digest long fat molecules. Without lipase, fat would pass quickly through our system, resulting in the possibility of diarrhea and sometimes leakage from the lower bowel. In addition, the human body relies on certain essential fatty acids that can only be derived from food and Lipase is used to cultivate those fatty acids.

Without Lipase, the human body cell structures cannot function normally and humans would also suffer from extremely dry skin and hair. Preferred amounts of lipase for the teachings herein include 25 mg or between 18-32 mg.

Protease. Protease is the general term for an enzyme that breaks down proteins. Proteins are molecules that make up much of our living tissue, including our muscles and our internal systemic enzymes.

Certain proteins can only be provided through our food and Protease assists in breaking down our food to cultivate those proteins. If the human body has an inadequate means of cultivating proteins; humans would suffer from what is known as “protein malnutrition”. Proteins are broken down in several steps. As most proteins are not simply long, skinny molecules, but rather coil-like, they must be “unraveled”. Much of this unraveling is done in the acidic environment of the stomach.

Generally, proteins do not tolerate an acidic environment unless specifically designed to do so. Once unraveled, proteases break down the pieces of the protein into amino acids that are easily absorbed into the human body. Much of the body wouldn't be able to function properly without essential amino acids from absorbable protein. A preferred protease that can be used with each embodiment herein relating to a protease is bromelain. teachings herein is bromelain, which refers to either of two protease enzymes extracted from the plants of the family Bromeliaceae. Preferred amounts of protease, such as bromelain, that can be used in the teachings herein include 18 mg or between 13-23 mg.

Maltase. Maltase is an enzyme that breaks down a specific sugar that is ingested by humans. The particular sugar is malt sugar, which is often found in malt liquor and other malted foods. Preferred amounts of maltase, that can be used in the teachings herein include 10 mg or between 8-12 mg.

Invertase. Invertase is also an enzyme that breaks down a specific sugar ingested by humans. That particular sugar is sucrose or table sugar. Those of us with a high sugar intake are in particular need of this enzyme. If invertase cannot do its job, the bacteria in our gut are left to attack the ingested sugars. Stomach cramps, bloating and gas can result if sugar-digesting enzymes are inadequate. Preferred amounts of invertase, that can be used in the teachings herein include 18 mg or between 13-23 mg.

Lactase. Lactase is another specific enzyme that breaks down sugars, particularly the sugar found in dairy products. Without lactase, our ability to drink milk or consume other dairy products would be greatly impaired. As with many enzymes, you don't have to be born with intolerance to lactose to have insufficient lactase levels. Any individual who quits consuming dairy products for a period of time may find that when they begin drinking milk again, they are suddenly intolerant. This is because the body gradually “forgets” to make an enzyme that isn't getting used much. In this case, lactase supplementation may become necessary later in life even though an individual has not previously had a problem with lactose intolerance. Preferred amounts of lactase that can be used in the teachings herein include 9.5 or between 8.0-11 mg.

Papain. Papain, also known as papaya proteinase I, works in the digestive system to break up specific segments of proteins into smaller amino acids. Specifically, papain is beneficial for breaking down tough meat fibers. Preferred amounts of papain that can be used in the teachings herein include 1.7 mg or between 0.5-3 mg.

Hemicellulase. Hemicellulase is an enzyme that is vital to the digestion of plant material. Plant cell walls are made from cellulose and are often difficult to digest. Poor plant digestion leaves an excess of roughage that is eaten by bacteria in the colon. The end result is gas and sometimes intolerance to raw vegetables. Hemicellulase keeps this intolerance from happening and maximizes the nutrients that can be absorbed from raw vegetables. Preferred amounts of Hemicellulase that can be used in the teachings herein include 8 mg—or between 5-11 mg.

Glucoamylase. Glucoamylase is a saccharide digestive enzyme. It is found mostly in mucosa and its function is to assure the breakdown of maltose into glucose molecules. Preferred amounts of Glucoamylase that can be used in the teachings herein include 50 mg or between 30-70 mg.

Xylanase is a group of enzymes that break down components of the cell wall matrix of plants (fiber), such as hemicellulose. Although xylanase is not produced by humans, it is present in fungi from which it may be used for the degradation of plant matter into usable nutrients.

In one further aspect of the invention the composition may include at least one ingredient of each of the lactobacillus varieties noted above for at least 3×10⁹ colony forming units (at time of manufacture assuming half billion cfu per probiotic; and preferably 6×10⁹ colony forming units assuming 1 billion cfu per probiotic at time of manufacture), together with a digestive enzyme and contained in a vegetable-based capsule. Preferably the capsule is stored in a blister pack. Preferred amounts of Xylanase that can be used in the teachings herein include 3.9 mg or between 2-6 mg.

In one further aspect of the invention the composition may include at least one ingredient of each of the lactobacillus varieties and at least one ingredient of each of the Bifidobacterium varieties noted above for at least 4.5×10⁹ colony forming units (at time of manufacture assuming half billion cfu per probiotic; and preferably 9×10⁹ colony forming units assuming 1 billion cfu per probiotic at time of manufacture), together with a digestive enzyme and contained in a vegetable-based capsule. Preferably the capsule is stored in a blister pack.

In yet a further aspect of the invention the composition may include at least one of the probiotics of the lactobacillus variety and at least one probiotic of the Bifidobacterium variety together with a digestive enzyme and contained in a vegetable-based capsule for at least 2×10⁹ colony forming units. Preferably the capsule is stored in a blister pack. More preferably the blend includes at least some additional probiotic ingredients as noted above and at least 6×10⁹ colony forming units assuming 1 billion cfu per probiotic at time of manufacture. In a further and preferred aspect, the composition may include, for instance, Lactobacillus acidophilus, Lactobacillus rhamnosus CG, Bifidobacterium infantis, and Bifidobacterium bifidum, together with a digestive enzyme and contained in a vegetable-based capsule. In a further preferred aspect the foregoing composition may include others from the above list of probiotics for at least 9×10⁹ colony forming units, and stored in a blister pack or other sealed package. In a further preferred aspect the foregoing composition may include others from the above list of probiotics for at least 13×10⁹ colony forming units, and stored in a blister pack or other sealed package. In a further aspect the composition may include a greater amount of lactobacillus probiotic as compared to Bifidobacterium probiotic.

According to preferred embodiments, the total composition contains the following amounts of each probiotic: Bifidobacterium Infantis—6 billion cfu, Bifidobacterium Longum —1 billion cfu, Bifidobacterium Bifidum —4 billion cfu, Lactobacillus Rhamnosus —6 billion cfu, Lactobacillus Acidophilus —2 billion cfu, Lactobacillus salivarius —2 billion cfu, Lactobacillus plantarum—2 billion cfu, Lactobacillus Casei—1 billion cfu, Lactobacillus paracasei—2 billion cfu.

According to further embodiments, the total composition contains the following amounts of each probiotic: Bifidobacterium Infantis—between 5-7 billion cfu, Bifidobacterium Longum—between 750 million and 2 billion cfu, Bifidobacterium Bifidum—between 3-5 billion cfu, Lactobacillus Rhamnosus—between 5-7 billion cfu, Lactobacillus Acidophilus—between 1-3 billion cfu, Lactobacillus salivarius—between 1-3 billion cfu, Lactobacillus plantarum—between 1-3 billion cfu, Lactobacillus Casei—between 750,000 million-2 billion cfu, Lactobacillus paracasei—between 1-3 billion cfu.

According to preferred embodiments, the total composition contains the following amounts of each enzyme: hemicellulase—8 mg, xylanase—3.9 mg, amylase—1.33 mg, glucoamylase—50 mg, maltase—10 mg, papain—1.7 mg, protease, such as bromelain—18 mg, lipase—25 mg, invertase—1.5 mg, lactase—9.5 mg.

According to further embodiments, the total composition contains the following amounts of each enzyme: hemicellulase—between 5-11 mg, xylanase—between 2-6 mg, amylase—between 0.5-2.5 mg, glucoamylase—between 30-70 mg, maltase—between 8-12 mg, papain—between 0.5-3 mg, protease, such as bromelain—between 13-23 mg, lipase—between 18-32 mg, invertase—between 0.5-3 mg, lactase—between 8.0-11 mg.

According to further embodiments, the total composition contains the following amounts of each enzyme: hemicellulase—3,000 CU, xylanase—550XU, amylase—23,000 DU, glucoamylase—350 XU, maltase—200 DP, papain—50 AG, Protease, such as bromelain—80,000 HUT, lipase—3,500 FCCFIP, invertase—79 INVU, lactase—900ALU.

### EXAMPLES

### Example 1: Effects of a Probiotic/Digestive Enzyme Composition on Cholesterol Metabolism in a Mouse Model of Hypercholesterolemia

Description of Study and Methods:

A murine model of hypercholesterolemia induced by a high fat diet was used to test the influence of a probiotic/digestive enzymes supplement on blood cholesterol levels.

The dietary supplement used in these experiments consisted of capsules containing a blend of probiotics (total dry weight equaling 116.20 mg); specifically, Bifidobacterium infantis, Bifidobacterium bifidum, Lactobacillus acidophilus, Lactobacillus salivarius, Lactobacillus plantarum, Lactobacillus rhamnosus, Bifidobacterium longum, Lactobacillus casei, Lactobacillus paracasei, and digestive enzymes (dry weight equaling 272.65 mg); specifically, amylase, glucoamylase, lipase, bromelain, maltase, lactase, hemicellulase, xylanase, papain, and invertase. The following amounts of probiotics were used: Bifidobacterium Infantis—6 billion cfu, Bifidobacterium Longum —1 billion cfu, Bifidobacterium Bifidum —4 billion cfu, Lactobacillus Rhamnosus —6 billion cfu, Lactobacillus Acidophilus —2 billion cfu, Lactobacillus salivarius —2 billion cfu, Lactobacillus plantarum—2 billion cfu, Lactobacillus Casei—1 billion cfu, Lactobacillus paracasei—2 billion cfu.

In addition, the following amounts of enzymes were used: hemicellulase—8 mg, xylanase—3.9 mg, amylase—1.33 mg, glucoamylase—50 mg, maltase—10 mg, papain—1.7 mg, bromelain—18 mg, lipase—25 mg, invertase—1.5 mg, and lactase—9.5 mg.

C57BL/6J mice were randomly assigned to three groups: 1) Control mice fed a regular, low fat diet (4% fat content; n=10); 2) Control mice fed a high-fat diet (35% fat content; n=10); and, 3) Test mice fed the high fat diet (35% fat content) supplemented with the probiotic/digestive enzyme composition in their drinking water (n=10), which was prepared by dissolving the contents of the capsules in the water. The high fat diet was intended to simulate human diets enriched in fat and sugar and contained a fat content of 35.00% and a calorie content of 22.40 Kj/G, which contained beef tallow and hydrogenated vegetable shortening as the lipid components. The influence of the probiotic/digestive enzyme composition in mice fed this unhealthy diet could therefore be evaluated.

Mice were supplemented with the probiotic/digestive enzyme composition for eight weeks. Serum cholesterol concentrations were measured before supplementation with probiotics/digestive enzymes, after 4 weeks of supplementation, and after 8 weeks of supplementation with said composition.

Blood samples were taken by tail tipping at the time points indicated. Blood samples were collected in tubes and stored at 4° C. overnight and the sera were aspirated and stored in fresh tubes at −20° C. At the termination of the study, blood was also collected by cardiac puncture. Briefly, the mice were fasted for four hours, and then anesthetized with Ketamine (100 mg/kg body weight) and Xylazine (5 mg/kg) for the cardiac puncture procedure. Sera were harvested as described. Therefore, the data show measurements for sera taken from both peripheral blood (PB) and cardiac compartments.

Aliquots of serum taken from mice at the day 92, 120, and 148 time points of the trial were analyzed using biochemical assay kits for A total cholesterol (enzymatic cholesterol assay kit; XpressBio, Frederick Md., USA), LDL-C (colorimetric cholesterol assay kit; XpressBio), and triglycerides (enzymatic triglycerides assay kit; XpressBio). The concentration of HDL-C was calculated as the difference between total cholesterol and LDL-C.

Results and Conclusions:

Values for measurements of serum cholesterol are described below and are also provided in Tables 1 through 3 for LDL-C, HDL-C, triglycerides, respectively.

Low-density lipoprotein cholesterol (LDL-C), the fraction of serum cholesterol that is associated with atherogenesis and cardiovascular risk, was evaluated to determine whether the probiotic/digestive enzyme composition could have a desirable effect in lowering its concentrations in mice on a high fat diet. The values prior to supplementation with the probiotics/digestive enzyme composition were 23.64±6.01, 41.75±5.84, and 31.56±5.85 mg/dl in the low fat and the two high fat diet-fed groups, respectively (refer to Table 1). In mice given the supplement, four weeks of supplementation afforded a 34% decrease in serum LDL-C. Subsequently, within the group that was supplemented for eight weeks, there was a dramatic reduction of serum LDL-C from 31.56±5.85 mg/dl to 9.37±3.12 mg/dl as measured in peripheral blood. In serum taken by cardiac puncture after eight weeks, serum LDL-C was similarly low in supplemented mice (7.36±2.34 mg/dl). In fact, the serum LDL-C concentrations in the mice were 70.3% lower in peripheral blood than the level in the mice eight weeks earlier, and 78.5% lower than that in mice on the high fat diet that did not receive the probiotic/digestive enzyme supplement. These results demonstrate that the probiotic/digestive enzyme composition claimed herein affords significant and progressive reduction of harmful LDL-C levels in the circulation.

Assessment of high-density lipoprotein cholesterol (HDL-C), which is generally correlated with cardiovascular health, was measured as the difference between total cholesterol concentrations and LDL-C concentrations in serum for each time point (Table 2). No significant difference between the three groups could be appreciated between low and high fat diet groups before supplementation with the probiotic/digestive enzyme composition commenced. After four weeks of supplementation, HDL-C increased unexpectedly in the high fat diet group; however, a much greater increase in HDL-C was observed in supplemented mice. After four and eight weeks of supplementation, HDL-C concentrations measured in peripheral blood were 57.78±6.50 and 99.59±9.77, respectively, in mice supplemented with the composition, equaling a 72.4% increase over the course of the eight-week supplementation period. In serum samples taken by cardiac puncture, HDL-C levels were lower than those found in peripheral blood but followed the same trend of increasing in response to supplementation with the composition claimed herein.

Lastly, we investigated the influence of the probiotic/digestive enzyme composition claimed herein on triglycerides, a biomarker of cardiovascular risk, in mice fed a high fat diet. Triglyceride concentrations did not change overall in this mouse model (Table 3). After four weeks of supplementation with the probiotic/digestive enzyme composition, there was a modest reduction in triglyceride concentrations from 55.26±9.95 to 52.23±2.47 mg/dl; however, the result was not statistically significant. After another four weeks, triglyceride concentrations increased in all groups of mice examined but none of the changes were statistically significant. Mice supplement with the probiotics/digestive enzyme composition did no present with significant changes in triglyceride levels over the eight-week treatment course or in comparison to the unsupplemented group of mice fed the high fat diet.

The probiotic/digestive enzyme composition disclosed herein affords significant improvement in LDL-C lowering activity over other drugs/neutraceutical compounds previously tested in animal models of obesity and hypercholesterolemia. For example, in a mouse model of atherosclerosis, resveratrol (a natural phenol and phytoalexin) decreased plasma levels of LDL-C by 19%, comparable to atorvastatin (Lipitor®) alone (19%), and the combination of both agents decreased LDL-C by 22% over the 14-week study period. In another published study, LDL-C levels were reduced from 53% to 67% by ezetimibe (a cholesterol absorption inhibitor) treatment in a mouse model. For comparison, in this study, serum LDL-C concentrations in mice were >70% lower in peripheral blood than the level in the mice prior to eight-week supplementation with the probiotic/digestive enzyme composition disclosed herein (Table 1).

TABLE 1
Decreases in Low Density Lipoprotein Cholesterol Levels in Serum

| | LDL-C Concentrations (mg/dl) ± SEM | | |
|---|---|---|---|
| | | | High Fat Diet + |
| Treatment Time | Low Fat Diet | High Fat Diet | Supplement |
| Week 0 (PB) | 23.64 ± 6.01 | 41.75 ± 5.84 ∧ | 31.56 ± 5.85 |
| Week 4 (PB) | 28.91 ± 5.40 | 39.85 ± 4.64 | 20.88 ± 3.12 * |
| Week 8 (PB) | 20.59 ± 3.68 | 43.60 ± 5.38 ∧ | 9.37 ± 3.12 ∨ * |
| Week 8 (cardiac) | 15.27 ± 3.25 | 33.14 ± 6.35 ∧ | 7.36 ± 2.34 * |

* High Fat Diet vs. High Fat Diet + Probiotic/Digestive Enzymes is statistically significant difference (p < 0.05)
∧ Low Fat Diet vs. High Fat Diet
∨ Low Fat Diet vs. High Fat Diet + Probiotic/Digestive Enzymes
PB, serum from peripheral blood;
Cardiac, serum from blood obtained by cardiac puncture

TABLE 2
Increases in High Density Lipoprotein Cholesterol Levels

| | HDL-C Concentrations (mg/dl) ± SEM | | |
|---|---|---|---|
| | | | High Fat Diet + |
| Treatment Time | Low Fat Diet | High Fat Diet | Supplement |
| Week 0 (PB) | 46.45 ± 5.94 | 49.10 ± 4.22 | 57.78 ± 6.50 |
| Week 4 (PB) | 39.40 ± 4.61 | 58.59 ± 4.44 ∧ | 77.31 ± 11.65 ∨ |
| Week 8 (PB) | 43.93 ± 3.73 | 65.45 ± 5.36 ∧ | 99.59 ± 9.77 ∨ * |
| Week 8 (cardiac) | 43.04 ± 3.39 | 64.27 ± 5.46 ∧ | 82.98 ± 8.55 ∨ |

* High Fat Diet vs. High Fat Diet + Probiotic/Digestive Enzymes is statistically significant difference (p < 0.05)
∧ Low Fat Diet vs. High Fat Diet
∨ Low Fat Diet vs. High Fat Diet + Probiotic/Digestive Enzymes

TABLE 3
No Significant Change In Serum Triglycerides

| | Triglyceride Concentrations (mg/dl) ± SEM | | |
|---|---|---|---|
| | | | High Fat Diet + |
| Treatment Time | Low Fat Diet | High Fat Diet | Supplement |
| Week 0 (PB) | 46.98 ± 6.41 | 43.93 ± 1.24 ∧ | 55.26 ± 9.95 |
| Week 4 (PB) | 47.35 ± 1.62 | 49.28 ± 2.32 | 52.23 ± 2.47 |
| Week 8 (PB) | 57.97 ± 3.40 | 55.51 ± 3.51 | 57.80 ± 3.38 |
| Week 8 (cardiac) | 47.72 ± 4.28 | 51.80 ± 4.64 | 52.52 ± 4.69 |

∧ Low Fat Diet vs. High Fat Diet

### Example 2: Effects of a Probiotic/Digestive Enzyme Composition in the Simulator of Human Intestinal Microbial Ecosystem (SHIME®) System to Evaluate its Influence on Gut Microbiota

Description of Study and Methods:

The effects of the probiotics/digestive enzyme composition in the colon were evaluated using the well-established in vitro SHIME® system, colon reactors were operated for two weeks with “basal conditions” supporting the fermentation activity of gut microbiota (described below and known in the art), followed by three weeks of treatment with the probiotic/digestive enzyme composition to compare microbial compositions and short chain fatty acid production. The SHIME® system is known in the art as a means for evaluating changes in gut microbiota and their activity.

Colon reactors were inoculated with a fecal sample from a young adult donor and the experimental system was conducted as described previously. The SHIME® system consists of a series of double-jacketed vessels, simulating the digestive compartments. After inoculation, a two-week start up period was conducted to allow the microbial community to differentiate in the reactors. Subsequently, the reactors were run for a two-week period in which standard SHIME® feed was dosed to the system. The medium given consisted of arabinogalactan (1 g/L), pectin (2 g/L), xylan (1 g/L), starch (4.2 g/L), glucose (0.4 g/L), yeast extract (3 g/L), peptone (1 g/L), mucin (4 g/L), cysteine (0.5 g/L). This two-week feeding with medium established the baseline microbial community composition and activity in the different reactors and was considered the “Control Period”. Subsequently, in the “Treatment Period” the SHIME® reactor was operated under nominal conditions but including supplementation with the probiotic/digestive enzyme composition for three weeks. Supplementation was conducted using a sinker to accommodate 1 capsule of the probiotic/digestive enzyme composition in the stomach/small intestine compartment of the system. Each capsule contained blend of probiotics (total dry weight equaling 116.20 mg); specifically, Bifidobacterium infantis, Bifidobacterium bifidum, Lactobacillus acidophilus, Lactobacillus salivarius, Lactobacillus plantarum, Lactobacillus rhamnosus, Bifidobacterium longum, Lactobacillus casei, Lactobacillus paracasei, and digestive enzymes (dry weight equaling 272.65 mg); specifically, amylase, glucoamylase, lipase, bromelain, maltase, lactase, hemicellulase, xylanase, papain, and invertase.

Metabolic parameters were evaluated and compared in the SHIME® vessels during the two-week control period and the three-week treatment period of supplementation with the probiotic/digestive enzyme composition. The production of short-chain fatty acids (SCFA); specifically, propionate, was evaluated by standard gas-chromatographic methods from samples collected from the reactors weekly. SCFA are produced in the gut from fermentation of indigestible carbohydrates including dietary fiber, resistant starch, and oligosaccharides, and are absorbed from the colon into the liver as well as peripheral tissues. Production of SCFA is beneficial to numerous aspects of the digestive process including stimulation of the immune system and protection of the colon against cancer. Propionate in particular has been noted to decrease cholesterol synthesis in the liver, improving lipid metabolism. Lactate production was measured in the reactors to evaluate microbial metabolic activity. The composition of microbial communities, lactobacilli and bifidobacteria, were measured in each reactor by quantitative RT-PCR once weekly in the Control and Treatment periods. Since some strains of Bifidobacterium and Lactobacillus have been associated with improved cholesterol profiles and overall gut health, the influence of the probiotic/digestive enzyme composition disclosed herein was tested on simulated small intestine conditions.

Results and Conclusions:

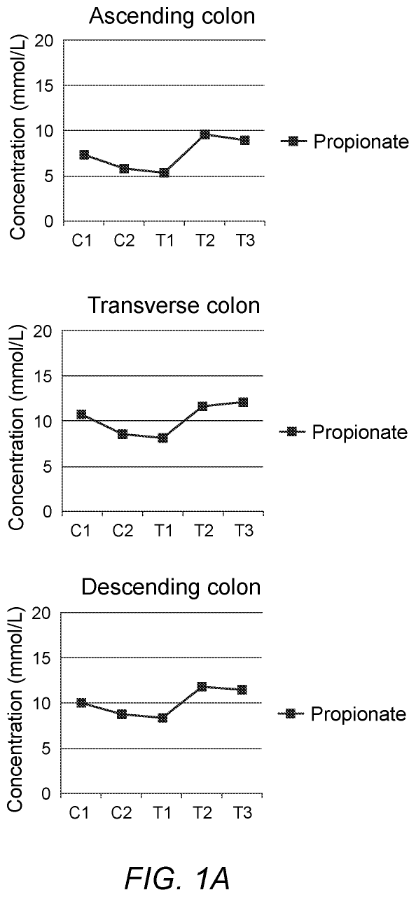

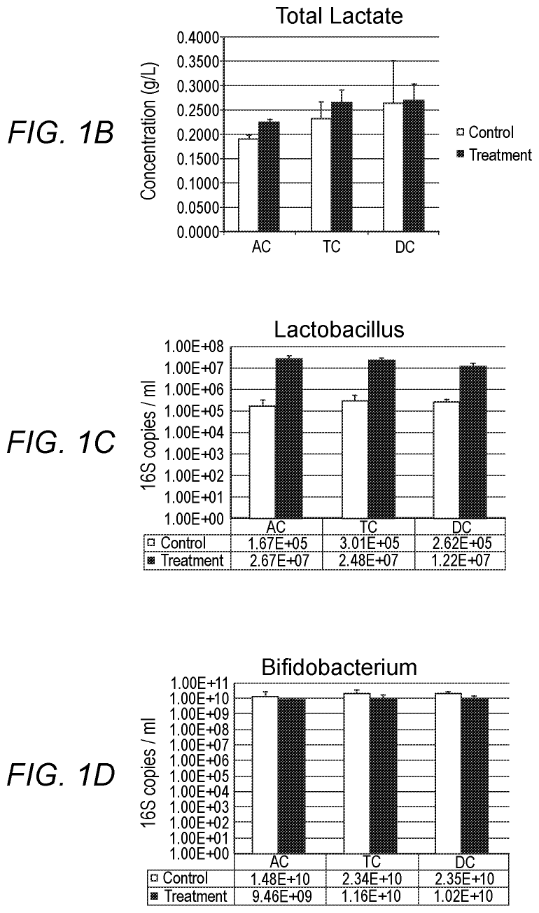

The results are summarized in FIG. 1. Briefly, addition of the probiotic/digestive enzyme composition to the in vitro system improved propionate production in the vessels corresponding to the ascending colon and transverse colon based on the comparison to the control (non-supplemented) two-week time period (FIG. 1A). An increased production of the metabolite lactate, a by-product of the fermentation process, was observed in the ascending and transverse colon reactors (FIG. 2B). Also, the presence of lactobacilli was increased in all the SHIME® reactor vessels (FIG. 1C), although no change in the bifidobacteria population was found in the supplemented system vs. the non-supplemented control vessels (FIG. 1D).

These results demonstrate survival and/or activity of probiotic bacteria from the probiotic/digestive enzyme composition under conditions that mimic the human gastrointestinal tract. The improved propionate production in supplemented vessels corresponding to the proximal colon supports a role for the probiotic/digestive enzyme composition as a modulator of fatty acid and cholesterol synthesis.

### Example 3: Case Study Evaluating the Effects of a Probiotic/Digestive Enzyme Composition on Cholesterol Profiles in Blood

A healthy volunteer was supplemented with 1 capsule per day of a probiotic/digestive enzyme composition disclosed herein. Each capsule contained a formulation of probiotics (116.20 mg total); specifically, Bifidobacterium infantis, Bifidobacterium bifidum, Lactobacillus acidophilus, Lactobacillus salivarius, Lactobacillus plantarum, Lactobacillus rhamnosus, Bifidobacterium longum, Lactobacillus casei, Lactobacillus paracasei, and digestive enzymes (272.65 mg total); specifically, amylase, glucoamylase, lipase, bromelain, maltase, lactase, hemicellulase, xylanase, papain, and invertase. The period of supplementation spanned from April until September 2015. As shown in Table 4, blood panel results from blood draws taken in April (prior to supplementation) vs. September (following daily supplementation), revealed a reduction in harmful triglycerides and LDL-C, as well as increased HDL-C in this individual. No changes in total cholesterol were observed. These results demonstrate that the probiotic/digestive enzyme composition can influence blood cholesterol profiles.

TABLE 4
Case Study of Blood Cholesterol Profiles In Response
to Probiotic/Digestive Enzyme Supplementation

| TEST (in mg/dl) | April | September |
|---|---|---|
| Triglycerides | 234 | 150 |
| HDL-C | 29 | 39 |
| LDL-C | 160 | 108 |
| Total Cholesterol | 209 | 223 |

The terms and descriptions used herein are set forth by way of illustration only and are not meant as limitations. Those skilled in the art will recognize that many variations of the inventive compositions and methods are possible within the spirit and scope of the subject matter of this invention. All terms of this inventive subject matter specification are to be understood in their broadest possible sense unless otherwise and specifically indicated.

What is claimed:

1. A pharmaceutical composition comprising: a formulation of probiotics comprising Bifidobacterium infantis, Bifidobacterium bifidum, Lactobacillus acidophilus, Lactobacillus salivarius, Lactobacillus plantarum, Lactobacillus rhamnosus, Bifidobacterium longum, Lactobacillus casei, and Lactobacillus paracasei; and, a formulation of digestive enzymes comprising: amylase, glucoamylase, lipase, bromelain, maltase, lactase, hemicellulase, xylanase, papain, and invertase.

2. (canceled)

3. (canceled)

4. (canceled)

5. The pharmaceutical composition of claim 1, wherein said formulations of probiotics and digestive enzymes are contained in a plurality of capsules.

6. The pharmaceutical composition of claim 5, wherein said formulations of probiotics and digestive enzymes are present in the same, single, capsule.

7. The pharmaceutical composition of claim 1, wherein said formulation of probiotics comprise probiotics in the following amounts: Bifidobacterium Infantis—between 5-7 billion cfu, Bifidobacterium Longum—between 750 million and 2 billion cfu, Bifidobacterium Bifidum—between 3-5 billion cfu, Lactobacillus Rhamnosus—between 5-7 billion cfu, Lactobacillus Acidophilus—between 1-3 billion cfu, Lactobacillus salivarius—between 1-3 billion cfu, Lactobacillus plantarum—between 1-3 billion cfu, and Lactobacillus Casei—between 750,000 million-2 billion cfu, Lactobacillus paracasei—between 1-3 billion cfu.

8. The pharmaceutical composition of claim 7, wherein said formulation of probiotics comprise probiotics in the following amounts: Bifidobacterium Infantis—6 billion cfu, Bifidobacterium Longum —1 billion cfu, Bifidobacterium Bifidum —4 billion cfu, Lactobacillus Rhamnosus —6 billion cfu, Lactobacillus Acidophilus —2 billion cfu, Lactobacillus salivarius —2 billion cfu, Lactobacillus plantarum—2 billion cfu, Lactobacillus Casei—1 billion cfu, Lactobacillus paracasei—2 billion cfu.

9. The pharmaceutical composition of claim 1, wherein said formulation of digestive enzymes comprise enzymes in the following amounts: hemicellulase—between 5-11 mg, xylanase—between 2-6 mg, amylase—between 0.5-2.5 mg, glucoamylase—between 30-70 mg, maltase—between 8-12 mg, papain—between 0.5 —3 mg, protease, such as bromelain—between 13-23 mg, lipase—between 18-32 mg, invertase—between 0.5-3 mg, lactase—between 8.0-11 mg.

10. The pharmaceutical composition of claim 9, wherein said formulation of digestive enzymes comprise enzymes in the following amounts: hemicellulase—8 mg, xylanase—3.9 mg, amylase—1.33 mg, glucoamylase—50 mg, maltase—10 mg, papain—1.7 mg, bromelain—18 mg, lipase—25 mg, invertase—1.5 mg, and lactase—9.5 mg.

11. A pharmaceutical composition comprising: (a) probiotic formulation comprising Bifidobacterium infantis, Bifidobacterium bifidum, Lactobacillus acidophilus, Lactobacillus salivarius, Lactobacillus plantarum, Lactobacillus rhamnosus GG, Bifidobacterium longum, Lactobacillus casei, Lactobacillus paracasei, and (b) a formulation of digestive enzymes.

12. The pharmaceutical composition of claim 11, wherein said formulation digestive enzymes are selected from the group consisting of: a) proteases; b) carbohydrate-digesting enzymes; c) Fiber-digesting enzymes; and, d) lipases.

13. The pharmaceutical composition of claim 12, wherein said carbohydrate-digesting enzymes are selected from the group consisting of: a) Amylase; b) Glucoamylase; c) Lactase; d) Invertase; and, e) Maltase.

14. The pharmaceutical composition of claim 12, wherein said fiber-digesting enzymes are selected from the group consisting of: a) Xylanase; and b) Hemicellulase.

15. (canceled)

16. The pharmaceutical composition of claim 11, wherein said formulations of probiotics and digestive enzymes are formulated into the same single formulation.

17. (canceled)

18. The pharmaceutical composition of claim 12, wherein said formulation of digestive enzymes comprises at least two proteases.

19. (canceled)

20. (canceled)

21. (canceled)

22. (canceled)

23. (canceled)

24. (canceled)

25. (canceled)

26. A method of treatment comprising: identifying a mammal in need of lowering of blood low-density lipoprotein cholesterol (LDL-C) and/or triglyceride concentrations, and administering to said mammal a pharmaceutical composition comprising:
(a) a probiotic formulation comprising: Bifidobacterium infantis, Bifidobacterium bifidum, Lactobacillus acidophilus, Lactobacillus salivarius, Lactobacillus plantarum, Lactobacillus rhamnosus, Bifidobacterium longum, Lactobacillus casei, Lactobacillus paracasei; and
(b) a formulation of digestive enzymes comprising: amylase, glucoamylase, lipase, bromelain, maltase, lactase, hemicellulase, xylanase, papain, and invertase;
in an amount sufficient to lower LDL C and/or triglycerides in the subject in need.

27. The method of claim 26, wherein said probiotic formulation and formulation of digestive enzymes are combined in an oral capsule.

28. (canceled)

29. The method of claim 26, wherein said probiotic formulation comprises probiotics in the following amounts: Bifidobacterium Infantis—between 5-7 billion cfu, Bifidobacterium Longum—between 750 million and 2 billion cfu, Bifidobacterium Bifidum—between 3-5 billion cfu, Lactobacillus Rhamnosus—between 5-7 billion cfu, Lactobacillus Acidophilus—between 1-3 billion cfu, Lactobacillus salivarius—between 1-3 billion cfu, Lactobacillus plantarum—between 1-3 billion cfu, Lactobacillus Casei—between 750,000 million-2 billion cfu, Lactobacillus paracasei—between 1-3 billion cfu.

30. The method of claim 29, wherein said probiotic formulation comprises probiotics in the following amounts: Bifidobacterium Infantis—6 billion cfu, Bifidobacterium Longum —1 billion cfu, Bifidobacterium Bifidum —4 billion cfu, Lactobacillus Rhamnosus —6 billion cfu, Lactobacillus Acidophilus —2 billion cfu, Lactobacillus salivarius —2 billion cfu, Lactobacillus plantarum—2 billion cfu, Lactobacillus Casei—1 billion cfu, Lactobacillus paracasei—2 billion cfu.

31. The use of claim 19, wherein said formulation of digestive enzymes comprises enzymes the following amounts: hemicellulase—between 5-11 mg, xylanase—between 2-6 mg, amylase—between 0.5-2.5 mg, glucoamylase—between 30-70 mg, maltase—between 8-12 mg, papain—between 0.5-3 mg, protease, such as bromelain—between 13-23 mg, lipase—between 18-32 mg, invertase—between 0.5-3 mg, lactase—between 8.0-11 mg.

32. The use of claim 31, wherein said formulation of digestive enzymes comprises enzymes the following amounts: hemicellulase—8 mg, xylanase—3.9 mg, amylase—1.33 mg, glucoamylase—50 mg, maltase—10 mg, papain—1.7 mg, bromelain—18 mg, lipase—25 mg, invertase—1.5 mg, and lactase—9.5 mg.
